(19) United States
(12) Patent Application Publication
(10) Pub. No.: US 2026/0240503 A1
(43) Pub. Date: Aug. 20, 2026

(54) AI - POWERED HEALTH MONITORING SYSTEM

(71) Applicant: AIVOT Al PRIVATE LIMITED, Mumbai (IN)

(72) Inventor: Alok Kumar TIWARI, Navi Mumbai, Raigad, Mumbai (IN)

(21) Appl. No.: 19/164,712

(22) PCT Filed: Mar. 9, 2024
(86) PCT No.: PCT/IN2024/050240

§ 371 Date: Sep. 12, 2025

(30) Foreign Application Priority Data
Mar. 14, 2023 (IN) 202321016900

(51) Int. Cl. A61B 5/00; A61B 5/024; A61B 5/029

(52) U.S. Cl. CPC A61B 5/7267; A61B 5/0077; A61B 5/02416; A61B 5/029; A61B 5/6898; A61B 5/7275; A61B 5/742

(57) ABSTRACT

An AI-powered health monitoring system is for monitoring health parameters of a user. The system relies on extracting RGB signals from a sequence of images of a region of the user's body, which is used to generate PPG signals. The generated PPG signals are used in combination with the weighted average of model (WAM) along with the user's height, body weight and age in order to determine the health parameters. These health parameters may be cardiac parameters of the user, such as the stroke volume, cardiac output and cardiac index. AIML techniques are used to analyse data and provide actionable insights to healthcare professionals. The system may also be used to provide tailored dietary advice and recommendations to the user.

### TECHNICAL FIELD

The present disclosure is directed, in general, towards a system and a method for AI powered health monitoring systems. The present disclosure particularly relates to system and a method for monitoring the health parameters of a user by utilising photoplethysmography (PPG) signals and red-green-blue (RGB) signals extracted from a sequence of images of the user, along with other physical attributes of the user such as height, body weight, age, sex etc.

### BACKGROUND

The advancement of health monitoring technology has led to the development of various methods and devices for monitoring and determining health parameters of users. These methods and devices include mobile and smart health monitoring devices. With the help of these health monitoring devices, various health parameters, such as heart rate, blood pressure, and oxygen saturation, etc., which are crucial indicators of a person's overall health status, can be calculated with great convenience. Traditionally, these parameters were measured using invasive methods or specialised medical equipment such as pulse oximeters, glucometer, and blood pressure monitors. However, such medical equipment is expensive, time-consuming, and often requires manual record keeping. Furthermore, some traditional devices such as ECG machines use adhesive electrodes, which are required to be attached to the chest of a human body in order to measure the cardiovascular data. As can be seen, these methods, devices and equipment can be inconvenient, uncomfortable, expensive, time-consuming, and often require the specialised assistance of medical professionals. Furthermore, individuals living in remote areas often encounter challenges when trying to access said methods and devices for monitoring and determining their health parameters due to the lack of availability of these methods or devices, or sometimes not having the professional expertise near them.

For example, conventional pulse oximeters can be used for measuring the health parameters such as heart rate, blood pressure, blood oxygen saturation etc., of a user. However, in order to measure the health parameters of a user, pulse oximeters are required to be attached to the skin of the user. A pulse oximeter comprises a light source, preferably a green LED, and a photodetector for detecting light that has been transmitted through the skin. This time-varying transmitted information is used to isolate a Photoplethysmography (PPG) signal. Since the pulse oximeter is directly attached to the fingertip, it limits the user's freedom to move and use his finger. Furthermore, PPG devices can be of two types: Transmissive PPG and Reflective PPG. Transmissive PPG involves placing the light source and detector on opposite sides of the measuring sites whereas Reflective PPG involves placing the light source and detector on the same side of the measuring site. The use of contact PPG sensors is not possible in cases where a patient has suffered from severe skin burns, infections, wounds or any other contagious diseases. The aforementioned limitations of contact PPG make the utilisation of contactless photoplethysmography (PPG) increasingly appealing for measuring and monitoring cardiovascular data.

Recently, non-contact remote photoplethysmography (rPPG) has been introduced for the measurement of health parameters. rPPG comprises a light source for illuminating the area of focus and a detector to capture changes in skin colour due to changes in blood flow volume underneath the skin. Since it is non-invasive and non-contact, it is generally well suited for medical as well as non-medical applications, making it ideal for continuous monitoring of health parameters without discomfort to the user.

Affordable consumer health devices have revolutionised the health monitoring industry, empowering individuals to monitor their health parameters using an array of devices, including but not limited to smartwatches, fitness trackers, and blood pressure monitors. Accompanying these devices, software solutions are frequently provided that enable users to track various health parameters over extended periods. By tracking their health parameters, users can gain a better understanding of their health trends and identify potential health problems early on. However, the accuracy of these devices can vary widely. The accuracy of the readings of said health parameters recorded by these devices is not always comparable to the accuracy of the readings obtained using traditional medical equipment. Accuracy of these consumer devices depends on the quality of PPG sensors, lighting conditions, skin tone, noise, external factors and also on the algorithms employed for analysing these signals. Another problem is that users of these devices often lack the knowledge and skills to correctly interpret the data provided by these devices, which can lead to unnecessary concern or delay in seeking medical attention. These devices also carry concerns about data privacy and security when they are used to regularly collect and process medical data pertaining to the user. However, these devices have proven to be useful in several circumstances, providing critical information for timely diagnosis as well as serving as a convenient and accessible way to monitor health parameters of users, especially under the guidance of a medical professional.

Various types of health monitoring systems that are available today provide users with ease, comfort, and accurate measurements of their health parameters. Healthcare professionals have thoroughly evaluated patients' health parameters measured via mobile and smart devices, comparing them to measurements obtained through traditional systems and methods. The findings reveal enhanced accuracy in real-time measurements when utilising mobile and smart devices. Furthermore, the accuracy of these devices is increasing with time, as they gain access to better technology, data, hardware and software.

Photoplethysmography (PPG) is one of the smart health monitoring methods used for assessing changes in the blood volume within a vascular tissue bed. The technique utilises a light source, such as a green LED to illuminate the peripheral tissue, wherein the optical radiation traverses through various layers of tissue, experiencing scattering and absorption before either being transmitted through or reflected from the tissue surface. A diffuse reflection component carries the information of PPG as it diffuses through the skin, whereas a reflection component is the one scattered by the surface of the skin. The reduced intensity of this attenuated optical radiation is then captured by a photo detector, manifesting as a voltage signal recognized as the photoplethysmogram (PPG). However, in view of the recent developments in technology, photoplethysmographic signals can be measured remotely using ambient light and a conventional consumer level video camera, using red, green and blue colour channels (Verkruysse et al., Optics Express, 2008, 16:21434-21445).

Furthermore, health monitoring methods for the estimation of heart rate and respiratory rate using an RGB camera have been known (Hassan et al., Biomed Optics Express, 2017, 8:4838-4854). The heart rate and the respiratory rate are estimated from the PPG and the respiratory motion. The method employs the green spectrum of the RGB camera to generate a multivariate PPG signal to perform multivariate de-noising on the video signal to extract the resultant PPG signal.

The PPG healthcare devices may use two types of known methods to measure PPG signals: contact method and non-contact methods. Conventional PPG based healthcare devices use contact methods to measure the PPG signals. Contact based methods were unable to achieve proper measurements of said health parameters on damaged skin such as over skin burns, wounds or ulcers. Furthermore, contact PPG sensors are unable to assess body areas where there is movement. In spot measurement, only pulse rate could be monitored due to the localised region over which the PPG contact sensor is attached. In addition to that, to stay in contact with the skin, the contact PPG sensors put pressure over the area in contact which leads to disruption in microcirculation of blood and can lead to an unpleasant experience for the user.

Additionally, current PPG healthcare devices are generally static and lack the means for real-time analysis. They are not equipped to handle the complexity and variety of data needed for comprehensive health monitoring. Advancement in signal processing techniques and machine learning algorithms have the potential to significantly improve the accuracy of PPG based healthcare devices for health monitoring. Traditional consumer devices work by physically contacting the finger tip for health parameters measurement. Also these devices are not connected to the cloud or any other data storage means, so previously measured health parameters cannot be used to predict future outcomes, for example, for the determination of any abnormal conditions using regression models.

U.S. Pat. No. 9,615,749B2 discloses a “Method of remote monitoring of vital signs” by detecting the PPG signal in an image of a subject taken by a video camera such as a webcam. The PPG signal is generated through auto-regressive analysis of ambient light reflected off a specific area of the subject's skin. Frequency components of the ambient light and aliasing artefacts resulting from the frame rate of the video camera are cancelled by auto-regressive analysis of ambient light reflected from a region of interest not on the subject's skin, e.g. in the background. This discloses the spectral content of the ambient light allowing identification of the subject's PPG signal. It also discloses that the heart rate, oxygen saturation and breathing rate of the user can be obtained from the PPG signal.

U.S. Pat. No. 11,259,710B2 discloses a “System and method for remote measurements of vital signs” which deals with the remote monitoring of vital signs through a sequence of skin intensity measurements captured from various skin areas of an individual. It operates by solving an optimization problem with a solver that identifies frequency coefficients of photoplethysmographic waveforms based on these intensity measurements. The solver aims to minimise the variance between the skin's intensity values reconstructed based on these coefficients and the actual measured intensity values, while also applying joint sparsity to these coefficients. Furthermore, an estimator is responsible for extracting the vital signs from the frequency coefficients obtained from the photoplethysmographic waveforms.

U.S. Pat. No. 10,143,377B2 discloses a “Method for single channel imaging measurement of dynamic changes in heart or respiration rate” for remotely measuring or monitoring one or more physiological parameters in a subject, such as blood volume pulse, heart rate, respiratory wave, or respiration rate. The methods include capturing a series of images of the subject, and processing the images to obtain physiological parameters of interest. These methods can be used to analyse single channel signals, including signals obtained from active night vision cameras. As a result, these methods can be used to measure or monitor one or more physiological parameters in both daylight and low-light conditions.

None of the systems that exist are able to calculate health parameters such as cardiac parameters of a user accurately, relying primarily on the RGB and PPG signals obtained from images of a user, along with certain physical attributes of the user. Considering the limitations described hereinabove, there is a need for an automated, well-designed and intelligent system that overcomes these issues by ensuring accurate determination of health parameters of a user, such as the cardiac parameters of the user, using the above described RGB and PPG signals along with other physical attributes of the user, while also safeguarding the privacy and security of the user.

### SUMMARY

An object of the present disclosure is to provide a system and a method to determine health parameters of a user, such as cardiac parameters, using Photoplethysmography (PPG) and RGB signals.

Another object of the present disclosure is to provide a system and a method that can determine health parameters of a user, such as cardiac parameters, by using PPG and RGB signals along with other attributes of the user such as age, sex, body weight, and height.

Another object of the present disclosure is to provide a system and a method that can measure various cardiac parameters by utilising already measured cardiac parameters, such as stroke volume, in addition to the PPG and RGB signals.

Another object of the present disclosure is to provide a system and a method that can enable continuous real-time measurement of health parameters, such as cardiac parameters, without physical contact or any form of invasive procedure.

Another object of the present disclosure is to provide accurate and personalised recommendations for dietary advice based on the measured and historical health data of the user as well as the other attributes of the user such as height, body weight, age and sex.

Another object of the present disclosure is to record and track the health parameters of the user over time. By tracking the health parameters, users can easily identify potential risks associated with their health as early as possible, thus making detection and prevention more effective and efficient.

Another object of the present disclosure is to provide real-time feedback and alerts to the users about any anomalies or irregularities detected in their health parameters, by the proposed health monitoring system, which may require medical attention.

The following information presents a simplified summary of the disclosure in order to provide a basic understanding of the present disclosure. This summary does not limit the scope of the present disclosure in any way. Its sole purpose is to summarise some of the concepts disclosed herein as a prelude to the more detailed description that is presented at a later stage.

In order to overcome the problem of inaccurate measurement of health parameters using PPG signals, the present disclosure discloses a health monitoring system using PPG and RGB signals, along with user profile data (like age, sex, body weight and height), for accurate measurement, calculation and analysis of health parameters of users. As per a preferred embodiment of the present disclosure, an RGB camera can be used to capture a sequence of images of a human face, or another part of a user's body, and a computer vision algorithm can be used to determine the Region-of-Interest (RoI) in the sequence of images. Alternatively, the sequence of images may be uploaded by the user, instead of being captured in real time, for the purposes of health monitoring. Artificial Intelligence and Machine Learning (AIML) based techniques, already known in the art, can be employed to accurately extract red-green-blue signal (RGB signal) from the sequence of images. The extracted RGB signal is used for generating a photoplethysmography (PPG) signal corresponding to the sequence of images. Such AIML techniques can be trained on large datasets to recognize and isolate PPG related patterns from the sequence of images. By employing such techniques on the temporal and spatial information present in the sequence of images, RGB signals and PPG signals can be accurately detected and extracted from different types of cameras. It is understood by a person skilled in the art that any reference to sequence of images of a user's face or any other region of the user's body includes reference to video or other forms of visual data/information of a user's face or any other region of the user's body.

The RGB signal, in the embodiments of the present disclosure, is used to detect the change in blood volume underneath the user's skin, in the RoI captured in the sequence of images. Each colour in the RGB signal is associated with a different wavelength of light. These varying wavelengths of light penetrate the skin to different depths, such that these lights are absorbed by the blood in different layers of tissue. The changes in the RGB signals are then used to generate the PPG signals for that RoI. Most modern cameras such as webcams and smartphone cameras are capable of capturing RGB signals. This makes it convenient to use such devices for remote non-invasive PPG based health monitoring, without the need for specialised medical equipment.

Additionally, conventional devices, such as pulse oximeters, tend to use single-colour channels, (for example, the green colour channel). RGB signals on the other hand facilitates the use of multi-colour channels that provide more information for the extraction of accurate PPG signals corresponding to the RoI of the user's body.

In the assessment of health parameters through PPG, computer vision plays a crucial role. A computer vision algorithm can be employed on sequence of images captured in real time or sequence of images that are uploaded by the user, to identify the face and define the corresponding Regions-of-Interest (RoIs) such as the forehead, cheek or nose. Such RoIs have a high supply of blood vessels that makes it easy to detect subtle changes in the skin colour caused by the heartbeat. Also, the skin at such Rols is thin, which leads to better penetration of light through the skin and results in a higher quality PPG signal.

Computer vision techniques employed in a preferred embodiment of the present disclosure may also use other techniques such as filtering and pixel-based processing in order to improve the quality of the PPG signals.

The accuracy of the measurement of health parameters of a user, such as cardiac parameters, also depends on multiple factors relating to the sequence of images themselves, including the frame rate of the camera used to capture the images/video of the user and illumination on the RoI.

Out of the two techniques (i.e. motion-based and intensity-based) that are commonly used to determine the PPG signal, a preferred embodiment of the present disclosure uses the intensity-based technique in order to determine the PPG signal. When using the intensity-based method, tiny changes in the colour of skin pixels on the face of a user, caused due to blood flow fluctuations, are tracked in order to generate the PPG signal. This PPG signal represents the blood flow pattern underneath the skin of the user.

The PPG signals, when used in the proposed health monitoring system, are valuable for deriving multiple health parameters, including (but not limited to) cardiac parameters such as stroke volume, cardiac output and cardiac index. These health parameters are determined using the PPG signals along with other attributes of the user. Peak detection involves identifying maximum or minimum value in the PPG signal, which corresponds to systolic and diastolic points in the PPG signal. Other characteristics of PPG signals, such as height, area, pulse width, maximum and minimum slope, can also be determined along with the peak detection. By determining various relationships between these characteristics, various health parameters can be accurately determined.

The analysis of a PPG signal may comprise the following steps: filtration, feature extraction, advanced analysis of extracted features such as analysis in the time and frequency domain, utilisation of AIML techniques to determine and classify the signal parameters and finally determination of the health parameters of a user, such as cardiac parameters. The step of filtration of a PPG signal may include the application of filters such as bandpass filters, median filters, butterworth filters etc., to remove noise. The step of feature extraction may include peak detection and morphological analysis.

The present disclosure discloses an RGB and PPG signal based health monitoring system that receives a sequence of images using non-contact non-invasive techniques. A preferred embodiment of the present disclosure utilises the information in the PPG signal(s) along with body weighted averages of model (WAMs), wherein the WAM is an average body weight factor specific to different health parameters (including cardiac health parameters such as stroke volume), calculated using statistical data of other users. The utilisation of WAM enables an improved and accurate determination of health parameters of users based on RGB and PPG signals obtained using non-invasive methods. Additional health parameters such as the cardiac output and cardiac index can also be determined by using the PPG signal, WAM for the health parameter along with other attributes of the user such as the height and body weight of the user.

In one embodiment of the present disclosure, the PPG signal is generated from the RGB signal using a method known as plane-orthogonal-to-skin. This method is used to enhance the accuracy of PPG signals obtained from skin and also to minimise the impact of motional artefacts.

In another embodiment of the present disclosure, an artificial intelligence (AI) model is trained on a dataset of multiple samples including training data and test data, to analyse health parameters. The said AI model employs techniques to process and derive PPG and RGB signals from the image data of the user. Furthermore, the said AI model is also trained on the extracted PPG and RGB signals along with user profile data such as age, sex, body weight and height, which in return enhances the accuracy of the measured health parameters.

In & preferred embodiment of the present disclosure the health monitoring system includes an image acquisition unit (which either captures the images of the user or to which the user uploads the images), a processing unit, a memory unit and a display unit. The image acquisition unit may comprise a smartphone camera, a laptop camera or a webcam using which the user may capture a sequence of images and/or a user interface using which the user may upload a previously captured video or a sequence of images. Alternatively, the image acquisition unit may be a user interface using which the user may upload a video or a sequence of images of a part of the user's body. The data and machine or software instructions are stored in the memory unit. Moreover, the processing unit is equipped with specialised electronics and appropriate circuitry, which when activated by the instructions kept in the memory unit, produce relevant outcomes. These outcomes are then utilised to assess and calculate health parameters of a user, such as cardiac parameters of the users.

Furthermore, following the calculation of health parameters, the system is designed to transcribe the user's health data into a digital format that is easy to read and understand (for example, in tabular form or as a report). It is also programmed to generate a smart medical record summary/report that simplifies the comprehension of both present and historical health, emphasising any past abnormal conditions or the risk of certain health problems conditions in the future. Consequently, it offers tailored health recommendations, dietary advice and prescriptions based on the health parameters determined using the proposed health monitoring system, the user's profile data such as age, sex, body weight, height along with the user's past health records.

The above described embodiments are exemplary and outline rather broadly, the features and technical advantages of the present disclosure in order that the detailed description of the present disclosure that follows may be better understood. Additional features and advantages of the present disclosure will be described hereinafter. It should be appreciated by those skilled in the art that the conception and specific embodiments disclosed may be readily utilised as a basis for modifying or designing other structures or processes for carrying out the same purposes of the present disclosure.

Other aspects of the embodiments of the present described herein will be better appreciated and understood when considered in conjunction with the following detailed description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof.

### BRIEF DESCRIPTION OF DRAWINGS

FIG. 1 shows a flow diagram, as per a preferred embodiment of the present disclosure, explaining the general steps involved in the calculation of health parameters of a user.

FIG. 2 shows a block diagram of a preferred embodiment of the proposed health monitoring system, that is used to calculate health parameters of a user.

FIG. 3 shows the internal functional components of the learning engine 302 of a processing unit 202 as per a preferred embodiment of the proposed health monitoring system.

FIG. 4A-4B shows the working process of the proposed health monitoring system along with the communication link between the computing device and the proposed health monitoring system.

FIG. 5A-5H shows the screenshots of a mobile application installed in the computing device of a user, wherein the tailored advice and recommendations are offered based on the determination of the various health parameters of the user made using the proposed health monitoring system.

### DETAILED DESCRIPTION

The implementation of the embodiments of the present disclosure is discussed in detail below. It should be understood, however, that the present disclosure provides a broad scope of inventive concepts that can be embodied in a variety of specific implementations. The specific embodiments discussed herein are merely illustrative of specific ways to implement the present disclosure and do not, in any manner, limit the scope of the present disclosure.

In the following description, numerous specific details are set forth in order to provide a thorough understanding of embodiments of the present disclosure. It will be apparent to one skilled in the art that embodiments of the present disclosure may be practised without some of these specific details.

If the specification discloses a component or feature that “may”, “can”, “could”, or “might” be included or have a characteristic, that particular component or feature is not required to be included or have the characteristic.

As used in the description herein and throughout the claims that follow, the meaning of “a”, “an” and “the” includes plural reference unless the context clearly dictates otherwise. Also, as used in the description herein, the meaning of “in” includes “in” and “on” unless the context clearly dictates otherwise.

Throughout this specification, the use of the word “comprise”, “contain” and “include”, and variations such as “comprises”, “comprising”, “contains”, “containing”, “includes”, and “including” may imply the inclusion of other elements, not specifically recited as well.

Exemplary embodiments will now be described more fully hereafter with reference to the accompanying drawings, in which exemplary embodiments are shown. This present disclosure may, however, be embodied in many different forms and should not be constructed as limited to the embodiments set forth herein. Moreover, all statements herein reciting embodiments of the present disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Numerous modifications, changes, variations, substitutions, and equivalents of the embodiments described herein will be apparent to those skilled in the art, without departing from the spirit and scope of the present disclosure.

In a preferred embodiment of the present disclosure health monitoring system for monitoring health parameters of a user using a sequence of images, captured using a camera or provided by the user, is disclosed. It is understood by a person skilled in the art that any reference to sequence of images of a user's face or any other region of the user's body includes reference to video or other forms of visual data/information of a user's face or any other region of the user's body. The proposed system relates to an improved and accurate determination of health parameters of a user, such as cardiac parameters, by utilising the RGB signals and PPG signals, obtained and extracted from the sequence of images, while also taking into account user's attributes and profile data (such as age, sex, body weight, height). The proposed system facilitates the monitoring of health parameters of a user using non-invasive techniques, while maintaining and improving the accuracy of the health parameters that are determined. The proposed system facilitates remote monitoring of patients which in turn reduces hospital visits, which in turn saves money as well as time.

In an embodiment of the present disclosure, the proposed health monitoring system is designed to analyse and process data gathered from a user to generate a smart medical record summary/report that simplifies the comprehension of both present and historical health, emphasising any past abnormal conditions or the risk of certain health problems/conditions in the future.

In another embodiment of the present disclosure, the proposed health monitoring system trains AIML models for analysis of the health parameters based on the RGB signals and PPG signals, combined with user attributes and profile data (such as age, sex, body weight and height), which improves the accuracy of determination of health parameters over time.

In another embodiment of the present disclosure, the proposed health monitoring system is designed to utilise predictive modelling and nutritional science to provide tailored recommendations to the user, wherein the tailored recommendations relate to dietary requirements of the user and can also be based on the user's profile data such as age, sex, weight, height along with the past health records.

A preferred embodiment of the present disclosure discloses a non-invasive method of determining different health parameters of a user by acquiring a sequence of images of the user's body, extracting an RGB signal from the sequence of images, generating a PPG signal from the RGB signal using different methods, and then calculating the health parameters of a user such as stroke volume, cardiac output and cardiac index. The sequence of images may be captured using a camera or it may be uploaded by the user, for the purposes of health monitoring.

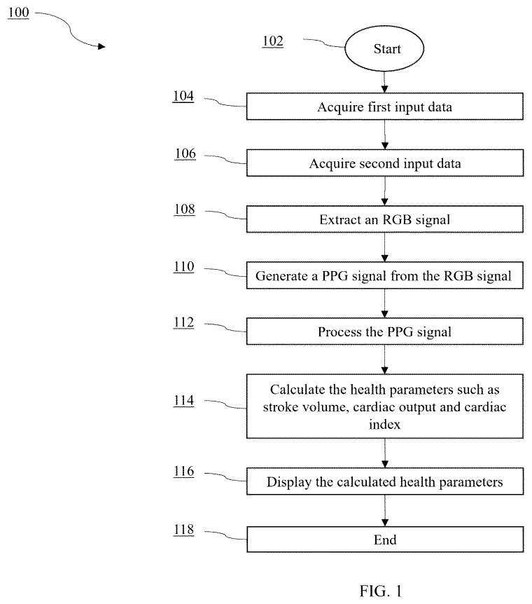

FIG. 1 provides a flow diagram, disclosing the steps involved in a preferred embodiment of the proposed health monitoring system, explaining the general steps involved in the calculation of the health parameters of a user 204.

As shown, a method 100 explaining the general steps involved in the calculation of health parameters of a user 204 is disclosed. The method starts 102 with initialisation of a device that contains the hardware as well as the computing capabilities to carry out the steps involved in practising the present disclosure.

The method 100 involves the acquisition 104 of first input data 212 comprising height, age and body weight of the user 204, which the user 204 can provide.

The second input data 208 is then acquired 106 using an image acquisition unit 206. The second input data 208 comprises a sequence of images of at least one region of the user's 204 body. As per a preferred embodiment of the present disclosure, this region of the user's 204 body is the user's 204 face. The sequence of images may be captured using an image capturing device like a camera. Alternatively, the sequence of images may be uploaded by the user 204. The image acquisition unit 206 may comprise a smartphone camera, a laptop camera or a webcam.

An RGB signal is extracted 108 from the second input data 208 by a processing unit 202.

A PPG signal 216 is generated 110 from the RGB signal 214, preferably using a method known as Plane-Orthogonal-to-Skin by using the processing unit 202.

The PPG signal 216 is processed 112 using various signal processing methods. These methods may include frequency filtering and peak detection.

The health parameters of the user 204 are calculated and determined 114 by using a combination of the processed PPG signal 216, the first input data 212 and a body weighted average of model (WAM) 210 of the respective parameters.

The health parameters are then displayed 116 to the user 204 using a display unit 232.

Once the calculated health parameters are displayed to the user 204 using a display unit 232, the user 204 may now close the user interface 240 installed in the computing device of the user 204, thereby ending 118 the method of calculation of health parameters of the user 204 by the proposed health monitoring system.

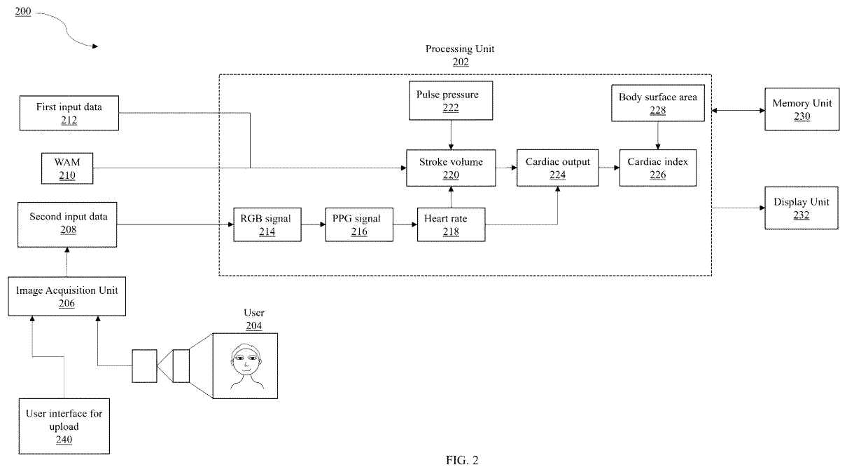

In accordance with a preferred embodiment of the present disclosure. FIG. 2 provides a block diagram of the proposed health monitoring system that is used to calculate the health parameters of the user 204.

The health monitoring system 200, as disclosed in a preferred embodiment of the present disclosure, comprises an image acquisition unit 206, a processing unit 202, a memory unit 230 and a display unit 232. The image acquisition unit 206 and the display unit 232 can be implemented as part of a single computing device such as smartphone, laptop, computer or tablet. They may also be implemented in separate devices. Alternatively, the image acquisition unit 206 may exist as a user interface 240, using which the user 204 may, among other things, upload a video or a sequence of images of a region of human's body. In a preferred embodiment of the present disclosure, the region of human's body is the human face. The processing unit 202 is communicatively coupled to the image acquisition unit 206 and the memory unit 230.

The image acquisition unit 206 and the display unit 232 are communicatively coupled to the processing unit 202. The processing unit 202 is also communicatively coupled to the memory unit 230. The memory unit 230 may be part of a server that is on-site, or in a remote location such as a cloud based server. The processing unit 202 includes suitable logic, circuitry, and/or interfaces that are operable to execute one or more instructions stored in the memory unit 230 to perform operations. The processing unit 202 can be of various architectures, some of the examples include, an x86 processor, a RISC processor, an ASIC processor and a CISC processor.

The image acquisition unit 206 is used to acquire a sequence of images, or a video, of a region of the user's 204 body-preferably the user's 204 face. The processing unit 202, after receiving the sequence of images, processes the received sequence of images to extract the quasiperiodic variation of the gradient intensity values of red, green and blue spectra of the visible light spectrum. Alternatively, a sequence of images may be uploaded by the user 204 using the user interface 240, instead of being captured in real time. The image acquisition unit 206 preferably has a minimum resolution of 620×480 pixels and is preferably capable of capturing the sequence of images at a rate of at least 30 frames per second. The resolution and frame rate, however, may vary based on different embodiments and implementations of the present disclosure.

The image acquisition 206 unit may include a smartphone camera, a laptop camera, a webcam or other similar devices. Alternatively, the image acquisition unit 206 may comprise a user interface 240 using which the user 204 can upload a video or a sequence of images for the purposes of health monitoring. In a preferred embodiment of the present disclosure, RGB cameras are used as they can capture the visible light spectrum. RGB cameras have three colour channels (red, green and blue) which allows them to capture different wavelengths of light. Generally, PPG only uses a single colour, often green, due to its higher absorption by the haemoglobin present in the blood. However, by using three colour channels namely red, green and blue (RGB), more data can be extracted from the RGB signals which in turn provides an accurate PPG signal to facilitate the accurate measurement of health parameters.

A user interface 240 comprising a software application or a web client can be used to acquire the first input data 212 such as height, age and body weight of the user 204. Moreover, the user interface 240 can be used to input data such as medical reports, x-rays etc. to the proposed health monitoring system for further analysis.

The PPG signal 216 is generated by measuring the variations in light intensity that occur beneath the surface of the skin. The PPG signal 216 can be isolated by performing multivariate de-noising of the RGB signal by using discrete wavelet decomposition or similar techniques that would be apparent to a person skilled in the art.

The first input data 212 comprises characteristics and physical attributes of the user 204. In a preferred embodiment of the present disclosure, the first input data 212 includes height, age and body weight of the user 204. The second input data 208, comprising the sequence of images of at least one region of the user's 204 body, acquired using an image acquisition unit 206, is received by the processing unit 202. In a preferred embodiment of the present disclosure, the second input data 208 comprises the sequence of images of the user's 204 face. After receiving the second input data 208, the processing unit 202 extracts the RGB signal 214 from the second input data 208, and generates a PPG signal 216 from the RGB signal 214. By conducting peak detection on the PPG signal 216, the user's 204 heart rate 218 is calculated.

The first input data 212, heart rate 218, pulse pressure 222 and another component known as body weighted average of model (WAM) 210 are required for calculating the user's 204 stroke volume 220 using the processing unit 202. Weighted Average of Model (WAM) 210 is the average body weight factor calculated using statistical data of a plurality of other users 204. WAM 210 is an average body weight factor specific to different health parameters (including cardiac health parameters such as stroke volume), calculated using statistical data of other users 204. The utilisation of WAM 210 enables an improved and accurate determination of health parameters of users 204 based on RGB signal 214 and PPG signal 216 obtained using non-invasive methods. Additional health parameters such as the cardiac output 224 and cardiac index 226 can also be determined by using the PPG signal 216, WAM 210 for the health parameter along with other attributes of the user 204 such as the height and body weight of the user 204.

The user's 204 cardiac output 224 is calculated by using the previously calculated stroke volume 220 of the user 204 and the heart rate 218 of the user 204. Additionally, the user's 204 cardiac index 226 is calculated by using previously calculated cardiac output 224 and body surface area 228 of the user 204.

The following equations are used to calculate the stroke volume, cardiac output as well as the cardiac index of the user, based on the WAM, first input data 212 as well as the extracted PPG signals:

$$Stroke Volume = [ ( WAM * W ) - ( WAM * A ) - ( WAM * HR ) + WAM ] * PP$$

wherein W=Body Weight of the user 204; A=Age of the user 204; HR=Heart Rate of the user 204; PP=Pulse Pressure of the user 204 and WAM=Weighted Average of model 210

$$Cardiac Output = [ \frac{( Stroke Volume * Heart Rate )}{1000} ]$$

$$Cardiac Index = [ \frac{Cardiac Output}{Body Surface Area} ]$$

$$Body Surface Area = WAM * Weight of the {user}^{0.425} * Height of the {user}^{0.725}$$

The heart rate 218 of the user 204 is calculated by conducting peak detection on the PPG signal 216 extracted from the RGB signal 214.

Pulse pressure 222 of the user 204 is calculated by determining the difference between systolic blood pressure and diastolic blood pressure, which are determined from the PPG signal using techniques already known in the art.

The processing unit 202 analyses the extracted health parameters and upon detection of any of the health parameters beyond a predefined threshold, the proposed health monitoring system transmits an alert signal to the computing device. The processing unit 202 may be designed in such a way that it is able to analyse health records i.e. medical reports, x-rays, or similar items either directly through the computing device or by uploading through the computing device to the memory unit 230. After receiving the health records i.e. medical reports, x-rays, or similar records/reports of the user 204, the processing unit 202 may be configured to convert the health records of users 204 into a digitally readable format that can be easily read and understood. Moreover, it may not only prepare a medical record view for easy understanding of historical health, but it may also emphasise any previous/prevailing abnormal conditions that may be determined by analysing such information and records. The proposed health monitoring system may also identify patterns and links of such conditions to their respective diagnoses and treatments, if available.

The computing device may be a smartphone, personal computer or any other similar electronic device. In an exemplary embodiment, information can be communicated between the proposed health monitoring system and the computing device i.e. the user 204 through the user interface 240. Additionally, the said user interface 240 may include a web client (e.g. a web browser) or a software application, which can be installed in the computing device.

The memory unit 230 is used to store different types of information and data including machine instructions, the first input data 212 comprising height, age and body weight of the user 204, the second input data 208 comprising a sequence of images of a region of the body of the user 204 and the processed data. The processing unit 202 executes machine instructions stored in the memory unit 230 to perform specific operations. Some of the most frequently utilised memory unit systems include, a Random Access Memory (RAM), a Read Only Memory (ROM), a Hard Disk Drive (HDD), and a Secure Digital (SD) card.

The display unit 232 may be implemented using several known technologies, such as (but not limited to) Cathode Ray Tube (CRT) based display, Liquid Crystal Display (LCD), Light Emitting Diode (LED) based display, Organic LED based display, and Retina display technology.

The proposed health monitoring system may be connected to a network that can be connected to in both wired as well as in wireless mode. In wired mode, interfaces such as an Ethernet port, a USB port or any other similar port can be employed. On the other hand, in wireless mode, an antenna can be employed to operate in accordance with various communication protocols, such as TCP, UDP, 2G, 3G, 4G, 5G or other communication protocols known in the art. Various devices in the system 200 can connect to the network in accordance with the various wired and wireless communication protocols such as TCP, UDP, and 2G, 3G, 4G, communication protocols.

Examples of the network may include, but are not limited to, a Wireless Fidelity (Wi-Fi) network, a Wide Area Network (WAN), a Local Area Network (LAN), or a Metropolitan Area Network (MAN). The network may also refer to the internet. Various devices in the system 200 can connect to the network accordance with the various wired and wireless communication protocols such as Transmission Control Protocol and Internet Protocol (TCP/IP), User Datagram Protocol (UDP), and 2G, 3G, 4G, 5G and other communication protocols.

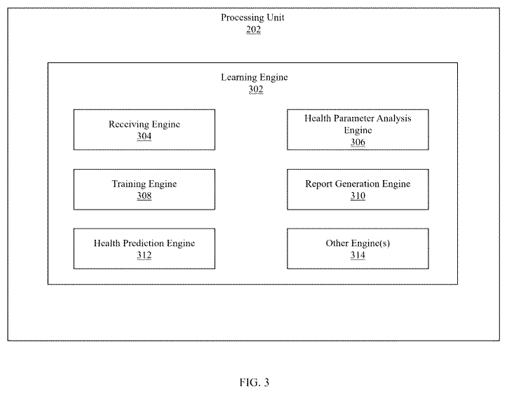

In accordance with a preferred embodiment of the present disclosure. FIG. 3 illustrates exemplary functional components of the learning engine 302 of the processing unit 202 of the proposed health monitoring system. The learning engine 302, as per a preferred embodiment of the present disclosure, is the most crucial component of the processing unit 202. The learning engine 302 can be realised as a combination of hardware as well as computing resources. For example, the hardware for the learning engine 302 may include a processor and the computing resources for the learning engine 302 may comprise machine readable instructions stored in non-transitory memory. In other examples, the learning engine 302 can be realised by using suitable electronic circuits.

In an embodiment, the learning engine 302 includes a receiving engine 304, a health parameters analysis engine 306, a training engine 308, a report generation engine 310, a health prediction engine 312, and other engine(s) 314. The other engine(s) 314 can implement functionalities that support applications or functions performed by system 200, the processing unit 202 or the learning engine 302. It would be appreciated that the modules/units being described are only exemplary and any other modules/units or sub-modules/sub-units may be included as part of system 200. These units too may be merged or divided into super-modules or sub-modules as may be desirable.

In a preferred embodiment of the present disclosure, the receiving engine 304, as part of the learning engine 302, may be designed such that it receives a sequence of images or video of a human face from an image acquisition unit 206. The receiving engine 304 may be set up to receive health records of the user 204, and also collect the personal characteristics and attributes of the user 204, such as age, body weight, sex, height, vitals, and historical health data.

In a preferred embodiment of the present disclosure, the main purpose of the health parameter analysis engine 306 is to analyse and process the received sequence of images of a region of the user's 204 body, preferably the face of the user 204. The health parameter analysis engine 306 uses suitable image analysis and AIML techniques to determine health parameters of the user 204, such as cardiac parameters of the user 204 which include stroke volume 220, cardiac output 224 and cardiac index 226. The health parameter analysis engine 306 may also be designed to analyse the extracted health parameters to identify any health parameters that exceed a certain limit or fulfil a certain criteria, in which case an alert signal through a network to the computing device of the user 204 to notify the user 204 of the same.

In a preferred embodiment of the present disclosure, the training engine 308 may be designed such that it receives the extracted health parameters and health records in machine-readable form or binary form. Additionally, the training engine 308 may be utilised to train the learning engine 302 using the health parameters that have been calculated. The training engine 308 is used to educate and optimise AIML models enabling it to process large amounts of data, identifying patterns and learning from them. In the proposed health monitoring system, the training engine 308 trains an AIML model in order to measure and train on various health parameters. The said AIML model is configured to train on the RGB signal 214, PPG signal 216 and other health parameters of the user 204 in order to improve the accuracy of measurements and calculations over time.

In a preferred embodiment of the present disclosure, the report generation engine 310 enables the intelligent conversion of the health records of users 204 into a digitally readable format (for ex. in PDF format, in tabular form or in graphical form). It may also prepare an intelligent and interactive medical record view for easy understanding as well as highlighting any past abnormal health condition, and providing diagnoses and prescriptions to assist a medical professional as well as the user 204. The report generation engine 310 may also use data analytics techniques to analyse, process and interpret health records, thereby generating a comprehensive and easily understandable medical record view.

In a preferred embodiment of the present disclosure, the health prediction engine 312 may be designed such that it provides accurate recommendations for tailored dietary advice based on the user's 204 profile, including age, body weight, physical activities, health parameters, and historical health data. Additionally, the health prediction engine 312 may use advanced predictive and forecasting techniques along with nutritional science to create personalised recommendations for macro and micro-nutrient intake, aiming to enhance the health and well-being of the user 204.

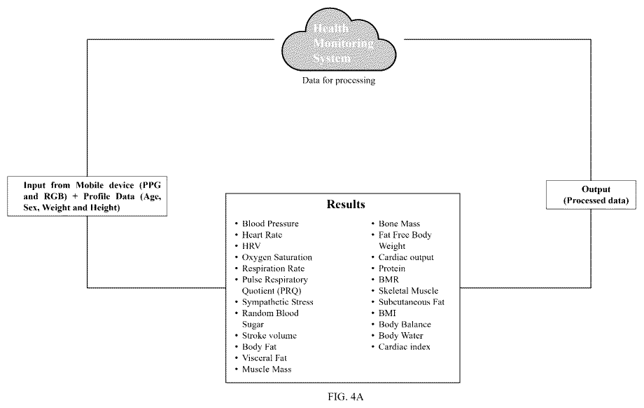

In accordance with a preferred embodiment of the present disclosure, FIG. 4 illustrates an exemplary working process of the proposed health monitoring system.

FIG. 4A and FIG. 4B reveal flow charts illustrating the high level process stages, starting from receiving information through the computing device, wherein the information may comprise the first input data 212 and the second input data 208 as the input, to the data being processed in the health monitoring system, and finally presenting the results in an appropriate format. The results provided in FIGS. 4A and 4B are exemplary. The present disclosure, may be practised in different ways to calculate different health parameters of the user 204.

The first input data 212 comprises attributes and characteristics of the user 204 such as height, age and body weight of the user 204. The first input data 212 is received from the computing device using a user interface 240 such as an application or a web client and the second input data 208 comprising a sequence of images of at least one region of the user's 204 body is received from a computing device. The received input data is then transmitted to the processing unit within the health monitoring system for further processing. The proposed system processes the data and produces outcomes i.e. values of health parameters such as stroke volume 220, cardiac output 224 and cardiac index 226.

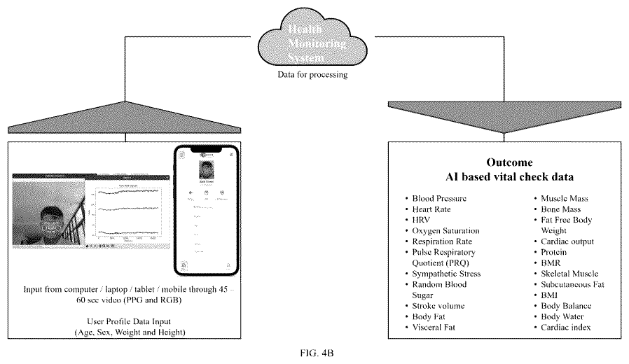

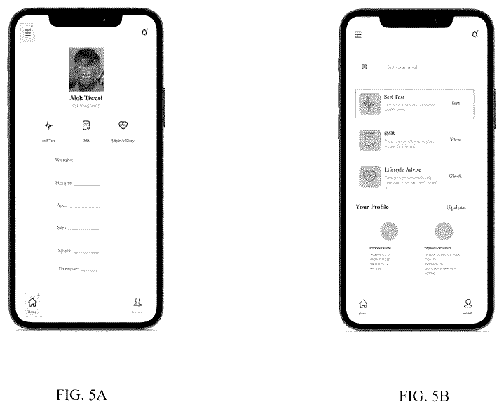

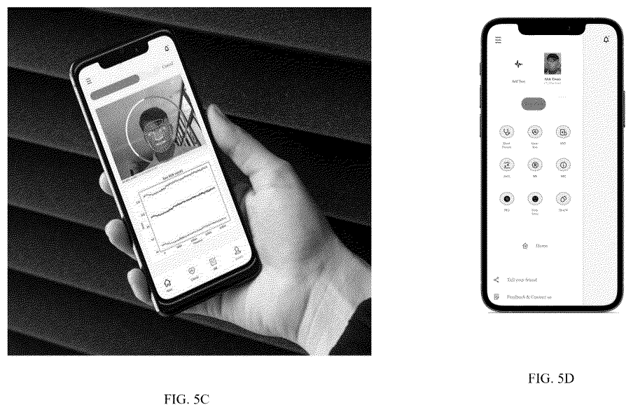

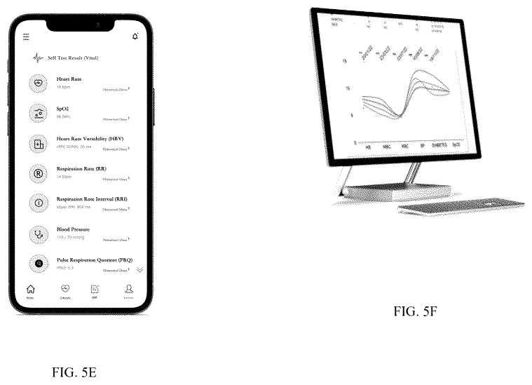

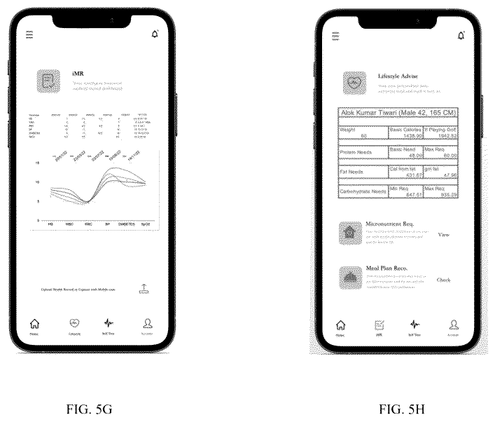

In accordance with a preferred embodiment of the present disclosure, FIG. 5 shows the screenshots of a mobile application installed in the computing device of a user 204. It is understood that FIG. 5, including all the screenshots shown from FIG. 5A to FIG. 5H are exemplary in nature and other user interfaces and methods may be used to receive information from the user 204 and display the results (including the health parameters) to the user 204.

FIG. 5A shows the user interface 240 where the user 204 provides his details comprising body weight, height, age, sex, sport, exercise or the like.

FIG. 5B shows the user interface 240 using which the user 204 may take a self-test, check iMR (i.e. intelligent medical records), and may receive lifestyle advice.

FIG. 5C-5E shows the user interface 240 using which the user 204 initiates a self-test, a sequence of images (which may be a video) of the user's 204 face is obtained, and the sequence of images are analysed to determine the health parameters, including cardiac parameters of the user 204 (such as stroke volume 220, cardiac output 224 and cardiac index 226), as described above in the embodiments of the present disclosure.

In FIG. 5F-5G, the proposed system 200 enables users 204 or healthcare professionals to see all the previous medical records in a visually appealing and interactive form. When diagnostic test reports, prescriptions or other medical records are uploaded by the user 204, the proposed health monitoring system 200 transforms that data into a tabular format and displays it on a dashboard in tabular and graphical form for easy understanding. Additionally, the system 200 detects any abnormal historical test data and highlights it as well as indicates it to a prescription or diagnostic report, if any. The system 200 provides clear and accurate information to healthcare professionals to diagnose more accurately and provide better treatment to patients.

In an exemplary embodiment, as shown in FIG. 5H, the proposed system 200 provides tailored dietary and meal planning recommendations based on the data it analyses, finding pinpoint areas of needed improvement for chosen goals of the users 204. Moreover, the system also provides macronutrient and micronutrient meal planning to the user 204. It empowers the users 204 to have a balanced, nutrient-dense diet based on their personal food preferences.

The programmable instructions of the proposed health monitoring system can be stored and transmitted on a computer-readable medium.

The proposed health monitoring system, in addition to being able to help medical professionals, can also support the utilisation of health data to aid the insurance industry in risk management, as well as in the processing of claims.

It is important to keep in mind the privacy and consent of the users when utilising such personal and medical data of users in industries such as insurance. It is imperative that informed consent of the users is taken for such applications and data is adequately anonymised and/or sanitized, as and when required. Safeguards must be implemented in order to prevent the misuse of personal and medical data of users and the applicable personal data protection laws must be complied with.

In an alternate embodiment of the present disclosure, the proposed health monitoring system may be used by insurance service providers for the purposes of risk management. Real-time health data of users may be gathered in an efficient and cost-effective way through smartphones, tablets, and laptops by the insurance service providers. Real-time health data has the potential to enhance risk evaluation, provide a personalised client experience, and empower the service provider to develop competitive offerings that match with the user's health needs. With the proposed health monitoring system, the onboarding and risk management can be significantly sped up by identifying high-risk clients based on their historical health records. The proposed health-monitoring system aims to enhance the user experience in insurance claims processing by leveraging health data to validate claims and minimise fraud. Overall, integrating the proposed health monitoring system into the insurance industry and ensuring that the privacy of users and their health and personal data is kept at the forefront, can offer a unified and trustworthy digital experience, reducing the need for medical examinations, and follow up claims.

The embodiments of the present disclosures disclose a non-invasive health monitoring system where the use of RGB and PPG signals eliminates the need for invasive procedures and specialised medical equipment. This allows for monitoring of health parameters without using invasive methods and techniques or other medical equipment.

The use of mobile cameras, laptop cameras or similar devices makes it convenient for users to monitor their health parameters at any time and from any location. The proposed system is also more affordable than traditional medical equipment, and also more accessible to a wider range of people.

The above described embodiments of the present disclosure are exemplary and non-limiting. They describe specific implementations of the present disclosure which are not to be construed as limiting the scope of the present disclosure. The present disclosure can be implemented in different manners and with modifications, which would be obvious to a person skilled in the art, without departing from the spirit and scope of the present disclosure.

What is claimed is:

1-14. (canceled)

15. A method for calculating one or more health parameters of a user, the method comprising:
acquiring a first input data comprising height, age and body weight of the user;
acquiring a second input data comprising a sequence of images of at least one region of a body of the user, wherein the sequence of images is acquired using an image acquisition unit;
extracting an RGB signal from the second input data using a processing unit;
generating a PPG signal from the RGB signal using the processing unit;
processing the PPG signal using the processing unit, wherein the processing unit is communicatively coupled to the image acquisition unit and a memory unit;
calculating a first health parameter of the user by using a combination of the processed PPG signal, the first input data and a first weighted average of model for the first health parameter, wherein the first health parameter is stroke volume of the user, and wherein the first weighted average of model is an average weight factor calculated using statistical data of a plurality of other users based on their historical stroke volumes; and
displaying the first health parameter user using a display unit.

16. The method as claimed in claim 15, wherein a second health parameter, being a cardiac output parameter of the user, is calculated using a heart rate of the user and the stroke volume of the user, and wherein the heart rate is calculated by conducting peak detection on the processed PPG signal.

17. The method as claimed in claim 16,
wherein a third health parameter, being a cardiac index parameter of the user, is calculated using the cardiac output parameter of the user and body surface area of the user,
wherein the body surface area of the user is calculated using a second weighted average of model for the third health parameter, height of the user and body weight of the user, and
wherein the second weighted average of model is an average weight factor calculated using statistical data of a plurality of other users based on their historical cardiac index parameters.

18. The method as claimed in claim 15, wherein the image acquisition unit used to capture the sequence of images comprises a webcam, a smartphone camera or a laptop camera.

19. The method as claimed in claim 15, wherein the at least one region of the user's body is a face of the user.

20. The method as claimed in claim 15, wherein plane-orthogonal-to-skin method is used to generate the PPG signal from the RGB signal.

21. A remote photoplethysmography system for calculating one or more health parameters of a user, comprising:
a user interface configured to receive a first input data comprising height, age and body weight of the user;
an image acquisition unit configured to acquire a second input data comprising a sequence of images of at least one region of the user's body;
a processing unit configured to receive the first input data and the second input data and calculate a first health parameter, the processing unit comprising a health parameter analysis engine,
the health parameter analysis engine being configured to extract an RGB signal from the second input data, generate a PPG signal from the RGB signal and calculate the first health parameter using a combination of the first input data, the processed PPG signal and a first weighted average of model, the first health parameter being a stroke volume of the user,
the first weighted average of model being an average weight factor calculated using statistical data of a plurality of other users based on their historical stroke volumes;
a memory unit storing one or more instructions, the first input data or the second input data; and
a display unit configured to display the one or more calculated health parameters of the user.

22. The remote photoplethysmography system as claimed in claim 21, wherein a second health parameter, being a cardiac output parameter of the user, is calculated using a heart rate of the user and the stroke volume of the user, and wherein the heart rate is calculated by conducting peak detection on the processed PPG signal.

23. The remote photoplethysmography system as claimed in claim 22, wherein a third health parameter, being a cardiac index parameter of the user, is calculated using the cardiac output parameter of the user and body surface area of the user,
wherein the body surface area of the user is calculated using a second weighted average of model, height of the user and body weight of the user, and
wherein the second weighted average of model is an average weight factor calculated using statistical data of a plurality of other users based on their historical cardiac index parameters.

24. The remote photoplethysmography system as claimed in claim 21, wherein the at least one region of the user's body is a face of the user.

25. The remote photoplethysmography system as claimed in claim 21, wherein the first input data, second input data and health parameters of the user are stored on a server, and wherein the server is an on-premise server, a remote server or a cloud based server.

26. The remote photoplethysmography system as claimed in claim 21, wherein the processing unit further comprises a learning engine.

27. The remote photoplethysmography system as claimed in claim 26, wherein the learning engine comprises:
a receiving engine configured to receive the sequence of images from the image acquisition unit;
a training engine configured to train the learning engine using the one or more calculated health parameters;
a report generation engine configured to convert medical records of the user into a digitally readable format, wherein the report generation engine is configured to analyze and interpret the medical records of the user; and
a health prediction engine configured to provide tailored recommendations to the user, wherein the tailored recommendations relate to dietary requirements of the user.

28. The remote photoplethysmography system as claimed in claim 26, wherein a database is used to store personal data and information of the user.
